(19) United States
(12) Patent Application Publication
(10) Pub. No.: US 2009/0191287 A1
(43) Pub. Date: Jul. 30, 2009

(54) Mitigation of Inflammation-Related Injuries

(76) Inventor: W. Dudley Johnson, Germantown, WI (US)

Correspondence Address: BOYLE FREDRICKSON S.C., 840 North Plankinton Avenue, MILWAUKEE, WI, 53203, US

(21) Appl. No.: 12/362,483

(22) Filed: Jan. 29, 2009

Related U.S. Application Data
(60) Provisional application No. 61/024,442, filed on Jan. 29, 2008.

(51) Int. Cl. A61K 31/519; A61K 36/00; A61K 36/736; A61K 31/05; A61K 31/12; A61P 29/00

(52) U.S. Cl. 424/735; 514/262.1; 424/725; 514/733; 514/679

(57) ABSTRACT

The present invention comprises formulae and methods that mitigate post-trauma inflammation, thus reducing overall damage to a patient. The formulae includes a uric acid agent, preferably a uric acid inhibitor, along with at least one other compound that is an inflammatory response suppressor. One preferred inflammatory response suppressor is a compound that reduces the omega 6/omega 3 EFA ratio in the body and preferably includes a source of omega 3 EFAs. The formulae are designed to minimize the formation of uric acid, which stimulates inflammatory response, and also minimize the production of ROS, an abundance of which is required for several inflammatory precursors or processes. A method of the present invention preferably includes prophylactic treatment of patients who are at risk for trauma, particularly traumatic brain injury, and includes daily administration of the selected formulation during the period of time in which risk is present.

### CROSS-REFERENCE TO RELATED APPLICATION

This application claims priority under 35 U.S.C. §119(e) to U.S. Provisional Application Ser. No. 61/024,442, filed Jan. 29, 2008 and entitled “Prevention or Minimization of Inflammation Related Neurological Injury,” the contents of which are hereby incorporated by reference in their entirety.

### FIELD OF INVENTION

The present invention relates to formulae and methods for reducing a patient's inflammation-related injury subsequent to a primary injury. Specifically, the invention relates to formulae and methods designed to reduce acute inflammation following injury that can be administered prophylactically.

### BACKGROUND

When bodily tissues experience a traumatic event, a generalized immune response in the form of tissue inflammation is generated. Inflammation assists the body in addressing the cause of trauma, and is beneficial in signaling further, directed immune response. However, the problems caused by inflammation may compound or overshadow the problem generated by the primary injury.

For example, it is known that brain damage, including traumatic brain injury, subsequent to a blunt head injury is primarily due, not to the injury itself, but to post-injury inflammation. Traumatic brain injury can cause severe, permanent brain damage, and is a leading cause of mental impairment in young people in industrialized countries due to auto accidents, sports-related injuries, and the like. Traumatic brain injury can also be caused by an ischemic episode, such as a stroke, in which blood flow to the brain is restricted. The physiological response to an ischemic stroke is identical to that of blunt injury response.

Similarly, other traumatic events may prevent blood from reaching various tissues. While blood flow stoppage can cause tissue damage, when circulation returns, reperfusion injury often occurs also resulting in tissue damage. A heart attack, for example, prevents blood from reaching the heart. When the heart attack has been treated, reperfusion of blood into the blocked areas of the heart often causes cell damage of a type called reperfusion injury that triggers inflammation. Again, the post-trauma “reperfusion” inflammation can cause permanent damage, and the deleterious long-term effects of heart damage can exceed the trauma of the heart attack itself.

In almost any situation where body tissues experience an impact injury or an ischemic event, reducing the inflammation that follows plays a significant role in recovery. Unfortunately, clinical treatment post-injury, especially following traumatic brain injury, has been largely unsuccessful, despite numerous therapeutic attempts. However, recent insights into the nature of inflammatory response suggest that preventative measures can mitigate inflammation, and thus mitigate the damage it causes.

Cell damage, such as is caused by direct trauma or lack of blood, causes the release of purines, which are metabolized by xanthine oxidase. The results of purine metabolization are uric acid and reactive oxygen species. Uric acid is known to excessively stimulate inflammatory response, and an abundance of reactive oxygen species allows additional inflammatory response, including (1) the accumulation of intracellular calcium, (2) the release of cytokines, and (3) arachidonic acid cascade.

The accumulation of calcium in cells contributes to their destruction and is often the first cause of cell death. Cell destruction generates additional reactive oxygen species, which can then contribute to further cell destruction. Cytokines are produced, such as IL-1, IL-6, and TNF-α, each of which signals for immune response, i.e. inflammation. Likewise, arachidonic acid is a precursor to pro-inflammatory signaling elements. Also, NF-κB (nuclear factor kappa-light-chain-enhancer of activated B cells) activates many genetic aspects of inflammation.

### SUMMARY OF THE INVENTION

The present invention is directed to formulae and methods that mitigate the inflammation that follows bodily trauma, thus reducing overall damage to a patient. The formulae are designed to minimize the formation of uric acid, which stimulates inflammatory response, and also minimize the production of reactive oxygen species (ROS).

Due to the clinical failure of post-traumatic therapies to mitigate inflammation, the method of the present invention preferably includes prophylactic treatment of patients who are at risk for trauma, and especially those at risk for traumatic brain injury. Military soldiers, law enforcement officers, players of high risk sports (football, downhill skiing, etc.), for example, would be potential candidates. The selected formulation is preferably dispensed for a period of time prior to the potential injury so that the active elements are present in the patient's bloodstream in effective quantities in the event of actual injury. The selected formulation is preferable taken daily during the period of time in which risk is present to provide continuous protection.

Furthermore, since some potential candidates are subject to risk of trauma for extended periods of time, such as in the case of a deployed soldier or law enforcement officer, the selected formulation should have no side effects, or very few, mild side effects. In addition, the formulation should preferably be relatively inexpensive for daily administration.

### DETAILED DESCRIPTION

Preferred formulations of the present invention include compounds that minimize uric acid and minimize the production of reactive oxygen species (ROS) in the body along with other inflammatory byproducts. The formulation may also include antioxidants, which help remove remaining ROS, as well as a TNF-α blocker.

As such, the present invention is directed to an orally ingestible formulation having a plurality of compound used to reduce the amount of acute inflammation that ordinarily occurs after a traumatic injury. Preferred formulations include a plurality of compounds that reduce the magnitude of the acute inflammatory response that occurs after a traumatic injury in mitigating the damage that the inflammatory response would ordinarily cause all while promoting a beneficial normal healing response. A particular and preferred use of one or more such formulations is for mitigating neurological injury to neurological tissue, including injury to brain tissue, where the injury is of a nature or location such that the acute inflammatory response would involve neurological tissue.

In at least one preferred formulation, there is a component that is a uric acid agent and at least one other component that reduces the inflammatory state or inflammatory potential in the body taking the formulation. In a preferred formulation, the uric acid agent inhibits uric acid formation. If desired, the uric acid agent can also be a compound that absorbs or otherwise neutralizes it in the body or a compound that causes the body to lower the amount of uric acid by excreting it.

At least one of the other compounds lowers the inflammatory state or potential by impacting one or more of the following inflammatory related processes or byproducts in a manner that reduces inflammatory response to a traumatic injury by affecting at least one of: (1) arachidonic acid cascade, (2) cell membrane permeability, (3) NF-κB (nuclear factor kappa-light-chain-enhancer of activated B cells), (4) free radicals, and/or (5) other inflammatory byproducts including Interleukin 1(IL1), Interleukin 6 (IL6), TNF-α and perhaps others. An example of one preferred compound that does this is a compound which reduces the omega 6/omega 3 essential fatty acid (EFA) ratio in the body of the person taking the formulation.

Such a formulation with this combination of compounds advantageously comprehensively reduces inflammatory response in the body of a person taking the formulation by reducing the magnitude of more than one type of inflammation, inflammation response, or inflammation trigger when a traumatic injury occurs. When taken in advance of a traumatic injury that would ordinarily trigger an undesirably excessive inflammatory response, the response is reduced or muted to such a degree that inflammation-related tissue damage is significantly reduced.

In one preferred formulation, the uric acid agent is a uric acid inhibitor and the at least one other compound is an inflammatory response suppressor, preferably a multiple inflammatory response suppressor, that preferably reduces the omega 6/omega 3 EFA ratio in the body. One preferred compound that inhibits uric acid formation upon the occurrence of a traumatic injury, such as a traumatic brain injury, is a compound that inhibits uric acid by blocking the enzyme xanthine oxidase. In a preferred formulation, the multiple inflammatory response suppressor is provided by at least one compound that (1) inhibits or blocks the arachidonic acid cascade that ordinarily occurs after the traumatic injury, (2) preserves post-injury cell membrane permeability of at least some of the cells in the region of the injury and surrounding the injury, (3) suppresses at least some formation of NF-κB, (4) absorbs and/or neutralizes free radicals, (5) inhibits or blocks some at least some Interleukin 1 and/or Interleukin 6 production, and/or (6) inhibits or blocks at least some TNF-α production. In one preferred formulation, the multiple inflammatory response suppressor is provided by at least one compound achieves at least a plurality of (1)-(6). In another preferred formulation, the multiple inflammatory response suppressor is at least one compound achieves all of (1)-(6). In a still further preferred formulation, such a multiple inflammatory response suppressor is comprised of a single compound. Where the formulation is intended for the mitigation of neurological tissue injury of the brain, at least one and preferably a plurality of the compounds of the formulation is of a type able to cross the blood-brain barrier.

### I. Uric Acid Agent

As discussed above, one component is a uric acid agent in an amount sufficient to impact uric acid by inhibiting its production, by absorbing or neutralizing it, and/or by excreting or otherwise expelling uric acid from the body. When taken in accordance with a prophylactic method of treatment using a formulation composed in accordance with the present invention, such a uric acid agent advantageously minimizes the impact of uric acid in the inflammatory response that occurs after dramatic injury.

In at least one formulation, the uric acid agent is allopurinol, which inhibits uric acid formation as explained in more detail below. In another formulation, other uric acid inhibitors can be used, such as Cat's Claw, also known as Uncaria tomentosa, or a cherry-based composition, such as preferably cherry juice or cherry juice extract. The uric acid agent can also be probenecid (trade name BENURYL®) or sulfinpyrazone (trade name ANTURANE®), which causes uric acid to be excreted from the body. If desired, a combination of one or more of these uric acid agents can be used.

Allopurinol is a known inhibitor of uric acid formation. It acts by blocking action of the enzyme xanthine oxidase, required for the breakdown of purines into uric acid. Use of allopurinol as a pretreatment has been studied in dogs. In one such study, the left anterior descending artery of subject dogs was ligated. In allopurinol-pretreated dogs, only 9% of the myocardium at risk was destroyed, while myocardial necrosis reached 23% in the control group. In another dog study, heart infarct size was 40% in control dogs and only 22% in dogs pretreated with allopurinol. In humans, in a double blind study on high-risk coronary surgery patients, one-half of patients received allopurinol before operating, while the others received a placebo. Those patients pretreated with allopurinol had a mortality rate of 4% while the control group, who received the placebo, had 17% mortality. As a side note, all of the patients who developed multi-organ failure were in the control group.

Allopurinol is well-suited for use in a preventive formulation because it has been used for many years in the treatment of gout such that its safety and efficacy is well known. For example, side effects are rare and typically limited to an allergic reaction when relatively high doses are taken. Thus, in one preferred formulation, an amount of allopurinol is included in the formulation to provide at least 0.25 mg per kilogram of body weight per day to a person taking the formulation on a daily basis. Thus, in another preferred formulation, an amount of allopurinol is included in the formulation to provide at least 1 mg per kilogram of body weight per day to a person taking the formulation on a daily basis. In another preferred formulation, the amount of allopurinol included in the formulation is enough to provide at least 2.5 mg per kilogram of body weight per day. In a currently preferred formulation, the amount of allopurinol included in the formulation is enough to provide at least 5 mg per kilogram of body weight per day. In at least one preferred formulation where allopurinol is used, the formulation includes at least 35 mg of allopurinol per day. In another preferred formulation where allopurinol is used, the formulation includes at least 300 mg all of allopurinol.

As previously discussed, it is contemplated that other uric acid inhibitors, such as Cat's Claw and cherry juice, e.g., cherry juice concentrate, can also be used. It is believed that both Cat's Claw and cherry juice concentrate are also effective inhibitors of uric acid formation by blocking the xanthine oxidase enzyme. Where Cat's Claw is included in the formulation, the amount of Cat's Claw included in the formulation is enough to provide at least 15 mg of Cat's Claw per day having a standardized formulation of at least 3% Uncaria tomentosa. In another preferred a formulation, the amount of Cat's Claw at least 30 mg. Where cherry juice concentrate is used, the amount may be determined by routine testing and experimentation. In addition to these other uric acid inhibitors, other types of uric acid inhibitors may be used such as other members of the plant families Rubiaceae or Rosaceae. It is anticipated that other natural inhibitors of uric acid formation will be discovered over time, and these will also be contemplated for use in the present invention.

Whether allopurinol, cat's claw, cherry juice concentrate, or another uric acid inhibitor is employed, the preferred formulation provides an amount of ingredient suitable to inhibit or otherwise minimize uric acid formation by building a high, stabilized level of a xanthine oxidase inhibitor in the patient's system. This minimizes uric acid formulation, which is released by dying cells as a danger signal, from sending additional danger signals which will stimulate further inflammation.

As also previously discussed above, the uric acid agent may be or include a compound that causes excretion or elimination of uric acid already present in the body of a person taking the formulation. Such uric acid agents include the uric acid excretion accelerator called probenecid (trade name BENURYL®) or sulfinpyrazone (trade name ANTURANE®) as these compounds increase uric acid in the urine, thus decreasing uric acid available in the body. For the reasons discussed above in relation to inhibition of uric acid production, a decrease in available uric acid helps reduce inflammation following traumatic injury including in particular traumatic brain injury. Naturally, it is preferable to block the formation of uric acid altogether rather than attempt to reduce its presence via excretion, but in certain cases a uric acid excretor or accelerator may be a valuable formulation addition.

### II. Inflammatory Response Suppressor

As also discussed above, a preferred formulation composed in accordance with the present invention includes at least one compound that is an inflammatory response suppressor that preferably is a multiple inflammatory response suppressor. One preferred multiple inflammatory response suppressor is a compound high in omega-3 EFAs, such as preferably fish oil or the like, which is advantageous and highly effective in reducing the omega 6/omega 3 EFA ratio in the body of the person taking the formulation for the reasons discussed below. Such omega-3 EFAs can be in the form all of alpha linolenic acid (ALA), eicosapentaenoic acid (EPA), docosahexaenoic acid (DH), or another form. Such a multiple inflammatory response suppressor preferably is prophylactically taken with the uric acid agent on a daily basis for at least a plurality of weeks prior to occurrence of traumatic injury.

In one preferred implementation, the formulation is a pill that includes at least one uric acid agent compound and at least one multiple inflammatory response suppressor compound. If desired, the uric acid agent compound and multiple inflammatory response suppressor compound can be separate pills or doses taken on the same day, such as at or about the same time. Of course, other types of oral delivery media methods and/or other types of oral ingestion and can be used.

The genetic evolution of humans corresponded with diets involving an intake of omega-6 to omega-3 fatty acids in an approximate ratio of 1:1. In modern times, many typical diets contain a ratio of omega-6 to omega-3 of closer to 20:1. An excess of omega-6 fatty acids increases inflammatory responses in humans because omega-6 fatty acids are the precursors to the release of cytokines, arachidonic acid cascades, and other thrombotic compounds.

Omega-3 fatty acid has several beneficial anti-inflammatory properties. First, omega-3 fatty acids preserve membrane function of damaged cells, and block the influx of calcium into damaged cells, the negative consequences of which have been previously described. Omega-3 fatty acids also prevent arachidonic acid cascade by (1) displacing available arachidonic acid in the system, (2) competing with arachidonic acid for critical enzymes; and (3) direct counteraction of arachidonic acid.

Omega-3 fatty acids also inhibit the effect of NF-κB, which is a cell-signaling molecule that signals for inflammation, and reduce the response of inflammatory products, including TNF-α, IL-1, and IL-6. Finally, omega-3 fatty acids provide raw material (DHA) for neuronal regeneration, essential in recovery from brain injury.

In one cat study, one group was pretreated with omega-3 fatty acids, in the form of fish oil, at 2% of its caloric intake for 3 weeks. Following ligation of the middle cerebral artery, the pretreated cats had 7% brain necrosis, while the untreated, control group, had 19% necrosis.

In a study involving human women, administration of fish oil at about 1% of daily caloric intake was performed for 3 months to both young women and older women. The ratio of arachidonic acid, an omega-6 fatty acid, to eicosapentaenoic acid, an omega-3 fatty acid, decreased from 10.6 to 2.5 in the younger women, and from 9.3 to 0.77 in the older women. Inflammatory products in blood (PgE2, TNF2, IL-6 and IL1B) were reduced 30-90%.

In another study, 18 grams per day of fish oil for three weeks suppressed IL-6 and TNF-α up to 61%. Finally, in a study of endothelial cells, DHA, another omega-3 fatty acid, reduced expression of inflammation (VCAM-1, IL-1, TNF2, IL-4) within 24 to 96 hours.

Omega-3 fatty acids are also advantageous in dampening inflammation as they reduce the release of TNF-α without entirely preventing release. This is beneficial since at least some TNF-α is required to enable sufficient signaling of the immune system so that a normal healing response is triggered. In addition, omega-3 fatty acids are powerful antioxidants, which bind with reactive oxygen species, thus making the reactive oxygen species unavailable for use in inflammatory signaling and processes. Furthermore, omega-3 fatty acids decrease cell permeability, thus preventing intracellular calcium influx. This reduces post-injury cell death, which in turn reduces the signaling of additional inflammatory response as well as reducing cellular release of purines that are broken into uric acid and reactive oxygen species.

Omega-3 fatty acids are found in dietary sources such as oily fish (salmon, mackerel, sardines, and the like), flax, walnuts, and so forth. It is contemplated that, for the purposes of providing prophylactic relief from trauma caused by inflammation, a supplement be administered in the amount of 1-2% of a patient's daily caloric intake. For example, for an athlete taking in 3,000 calories per day, the equivalent of 30-60 calories in omega-3 fatty acids (3-6 grams¹) are recommended. ¹ 1 gram fat=9 calories

One preferred formulation includes at least 500 mg of omega-3 fatty acids taken daily. Another preferred formulation includes at least about 1 gram of omega-3 fatty acids. A still further preferred formulation includes at least 2 grams of omega-3 fatty acids and preferably between 3-6 grams of omega-3 fatty acids. Where the source of omega-3 fatty acids is fish oil, a corresponding amount of fish oil is included in the formulation to achieve the aforementioned amounts of omega-3 fatty acids intake.

A formulation of the present invention preferably supplies a patient with a desirably high dose of omega-3 fatty acids, such as in the form of fish oil, to reduce their omega 6/omega 3 EFA ratio, such as by at least 10%, but it should be noted that the use of flaxseed oil, hemp oil, or any other suitable omega-3 fatty acid source would be well within the scope of the present invention. Furthermore, the provision of a dose of omega-3 fatty acid need not be from a naturally occurring source, and could be from a combination of sources. The source of omega-3 fatty acids to be used in the formulation may depend on considerations such as overall omega-3 fatty acid content, cost, purity, availability, etc.

### III. Antioxidant(s)

The formulation can also include one or more antioxidants. Antioxidants, as has been noted, bind with ROS, preventing the ROS from being employed in processes and signals required for additional inflammatory response. Preferred antioxidants include alpha lipoic acid, resveratrol, and turmeric or its primary antioxidant constituent, curcumin. Other antioxidants, or antioxidant precursors, such as selenium, can also be used.

Alpha lipoic acid, particularly dihydrolipoate, is a very effective scavenger ROS and is especially advantageous in cases of brain injury since it is one of the few antioxidants that is able to cross the blood-brain barrier. Alphoa lipoic acid is also able to enter cells and can therefore work both intra and extracellularly. It is a potent scavenger of hydrogen peroxide, a particularly toxic byproduct of oxidation. NFκB is rapidly inactivated by alpha lipoic acid.

In one preferred formulation, at least 1 mg per kilogram body weight per day is included in the formulation so as to be administered on a daily basis. In another preferred formulation, 5-10 mg per kilogram body weight per day is an easily administered dose that appears to be effective and is sufficient to maintain a stable blood level. One preferred formulation may contain about 500 mg or more of alpha lipoic acid. For example, one preferred formulation contains a daily dose of 700-1000 mg of alpha lipoic acid.

Although alpha lipoic acid is discussed in some detail, the disclosure of an antioxidant component to the formulation should not be considered limiting. A single antioxidant—beyond omega-3 fatty acids—or a mixture of different antioxidants may be determined to be most advantageous based on methods of administration, methods of production, product purity and efficacy, cost, availability, and the like. For example, while resveratrol is an antioxidant that is particularly well suited for use in a preventive neurological injury formulation, other antioxidants may be better suited for preventing other types of inflammation-related injury. In addition, it should be noted that the antioxidants can be synthetic or natural.

The antioxidant in the preferred formulation provides an amount of ingredient suitable to take up reactive oxygen species by building a high, stabilized level of antioxidant in the patient's bloodstream. The antioxidant should then be able to prevent or at least minimize the presence of ROS that increase inflammatory response and is typically produced after tissue trauma.

### IV. TNF Alpha Blocker

The formulation can also include TNF-α blocker. Where TNF-α blocker is used, routine testing and experimentation may be required to determine a suitable daily formulation amount.

TNF-α blockers prevent the action of tumor necrosis factor alpha (TNF alpha or TNF-α), which is a specific cytokine involved in inflammatory response. Blocking TNF-α prevents the induction of inflammation that would ordinarily be caused by its presence. Examples of suitable TNF-α blockers include adalimumab (trade name HUMIRA®), etanercept (trade name ENBREL®), and inflximab (trade name REMICADE®).

In brain injury, TNF-α is released directly from the injured brain and excess TNF-α is quite destructive. However, normal TNF-α levels are essential for healing after the first few hours post-injury. A TNF-α blocker may reduce initial injury, but blocking all TNF-α would interfere in subsequent healing.

Experimentally successful treatment to reduce traumatic brain injury has included injection of TNF-α blocker shortly after injury. However, the currently available TNF-α blockers are expensive and must be given intravenously. From a practical perspective, TNF-α blockers can not currently be used prophylactically. However, improvements in TNF-α blockers, including reduced cost and reduced toxicity, are anticipated in the future such that use of TNF-α blockers in the formulation of the present invention is contemplated.

It should be noted that omega-3 fatty acid, while not a TNF-α blocker, does effectively reduce elevated levels of TNF-α. In addition, it helps to maintain a normal functioning level of TNF-α, which has been shown to be essential for healing.

### V. Formulation

The formulation may be provided to the patient in one or more components. Preferably, all components selected for inclusion in the formulation will be combined for oral administration in a single tablet, capsule or liquid. However, a number of other ways to administer the formulation are acceptable and contemplated as within the scope of the invention. For example, the various components may be in separate tablets or capsules to better provide an individual patient with proper dosage based on his or her daily caloric intake and body weight. The formulation is preferably administered daily, but can also be administered in multiple doses with one or more constituents in one dose and one or more other constituents in another dose. For example, in one preferred formulation, allopurinol is given in tablet form separate from a pill or capsule containing an omega-3 fatty acid. Where the formulation also includes an antioxidant or antioxidant mixture that is different than an omega-3 fatty acid, the antioxidant or antioxidant mixture can be included in the omega-3 fatty acid containing pill or capsule.

There are hundreds of thousands of plant products in the world, a huge number of which have potential usefulness to humans. Additional products are likely to be identified in the future that have antioxidant or other anti-inflammatory effect, perhaps in even higher levels. Is it anticipated that the components used in the formulation of the present invention will be modified to take such discoveries into account.

### VI. Method

The term “prophylactic” treatment is art-recognized and refers to administration of a formulation prior to clinical manifestation of a problem, while “therapeutic” treatment occurs after manifestation of a problem. In a preferred method of the present invention, the formulation of the present invention is administered prophylactically.

A person at risk for trauma begins taking the formulation several days prior to the risk-event, and takes a daily dosage each day up to and including the day of risk. The formulation may be discontinued after the risk is over, or, in the event of continued risk, may continue taking the formulation. In the case of a football player, for example, the player might begin taking the formulation several days before the first practice of the season, and continue taking the formulation throughout the football season. However, at the end of the football season, when only routine strength-training or other non-contact fitness activities are expected, the player may discontinue taking the formulation.

The formulation should be prepared such that it contains enough of each element to achieve a stabilized level within the body of the patient that is suitably protective after taking the formulation at least a plurality of days. Three or more days of daily doses is the best known method of achieving a suitably protective level of elements at this time.

The components in the formulations discussed and claimed advantageously work together in a synergistic manner to prevent or minimize tissue damage from taking place after tissue injury by complementing one another, by significantly reducing but not completely eliminating TNF-α, by preventing or at least minimizing reperfusion related tissue damage, by reducing reactive oxygen species in the region of the injury, minimizing cell death by reducing and/or blocking cell permeability, and so forth to an extent or magnitude greater than that each components the facts are considered separately. In addition, where taken as a prophylactic or preventive treatment regimen, the formulations discussed and claimed also not only prevent or minimize post-injury tissue damage. Thus, even where neurological injury has occurred, the formulations disclosed in this application advantageously also protect the rest of the body thereby minimizing post injury tissue damage anywhere in the body. Finally, the formulations disclosed in this application prophylactically help prevent multi-organ failure and/or post-injury multi-organ failure or damage such that the formulations disclosed in this application are versatile, inexpensive, well-tolerated, have a minimum side effects, are easy to take, and provide benefits beyond post-injury tissue damage prevention.

It should be noted that the embodiments described herein explain the best known mode of practicing the invention and will enable others skilled in the art to utilize the invention, but are not to be considered limiting. Rather, it should be understood that the invention is not limited to the details of construction and arrangements of the components set forth herein, but is capable of other embodiments and of being practiced or carried out in various ways, and all such modifications and variations are within the scope of the claims set forth below. Further, various elements or features discussed herein may be combined in ways other than those specifically mentioned, and all such combinations are likewise within the scope of the invention.

I claim:

1. A formulation for minimizing inflammation related damage from a traumatic injury to a person comprised of at least one uric acid agent compound and at least one compound that lowers the inflammatory potential of the person taking the formulation.

2. The formulation of claim 1, wherein the uric acid agent comprises a compound that inhibits uric acid production.

3. The formulation of claim 2, wherein the uric acid agent comprises a compound that blocks xanthine oxidase enzyme action.

4. The formulation of claim 3, wherein the uric acid agent comprises allopurinol.

5. The formulation of claim 3, wherein the uric acid agent comprises Cat's Claw.

6. The formulation of claim 3, wherein the uric acid agent comprises cherry juice or cherry juice extract.

7. The formulation of claim 3, wherein the compound that lowers inflammatory potential comprises a compound that reduces the omega 6/omega 3 essential fatty acid ratio of the person taking the formulation.

8. The formulation of claim 7, wherein the compound that lowers inflammatory potential comprises a source of omega 3 fatty acids.

9. The formulation of claim 8, wherein the compound that lowers inflammatory potential comprises fish oil.

10. The formulation of claim 1, wherein the at least one compound that lowers inflammatory potential comprises an inflammatory response suppressor that affects at least one of (1) arachidonic acid cascade, (2) cell membrane permeability, (3) NF-κB, (4) free radicals, (5) Interleukin 1 production, (6) Interleukin 6 production, and (7) TNF-α production.

10. The formulation of claim 9, wherein the inflammatory response suppressor comprises a source of omega 3 fatty acids.

11. The formulation of claim 10, wherein the inflammatory response suppressor further comprises an antioxidant.

12. The formulation of claim 11, wherein the antioxidant comprises alpha lipoic acid.

13. The formulation of claim 11, wherein the antioxidant comprises one of resveratrol and curcumin.

14. The formulation of claim 11, wherein the inflammatory response suppressor further comprises a TNF-α blocker.

15. The formulation of claim 1, wherein the at least one compound that lowers inflammatory potential comprises a multiple inflammatory suppressor that does a plurality of the following in response to a traumatic injury: (1) inhibits or blocks arachidonic acid cascade, (2) preserves cell membrane permeability of at least some cells in the region of the dramatic injury, (3) suppresses formation of at least some NF-κB, (4) neutralizes free radicals, (5) inhibits or blocks Interleukin 1 production, (6) inhibits or blocks Interleukin 6 production, and (7) inhibits or blocks TNF-α production.

16. The formulation of claim 15, wherein the multiple inflammatory response suppressor comprises a source of omega 3 fatty acids.

17. The formulation of claim 16, wherein the source of omega 3 fatty acids comprises fish oil.

18. The formulation of claim 16, wherein the multiple inflammatory response suppressor further comprises an antioxidant.

19. The formulation of claim 18, wherein the antioxidant comprises an antioxidant that is able to cross the blood brain barrier.

20. The formulation of claim 18, wherein the antioxidant comprises alpha lipoic acid.

21. The formulation of claim 18, wherein the antioxidant comprises one of resveratrol and curcumin.

22. The formulation of claim 16, wherein the multiple inflammatory response suppressor further comprises a TNF-α blocker.

22. The formulation of claim 16, wherein the multiple inflammatory response suppressor further comprises a TNF-α blocker.

23. The formulation of claim 16, wherein the formulation is used to treat traumatic brain injury.

24. A formulation for minimizing inflammation related damage from a traumatic injury to a person comprising:
(a) an inhibitor of uric acid formation and (b) an omega-3 fatty acid.

25. The formulation of claim 24, wherein the inhibitor of uric acid formation comprises allopurinol.

26. The formulation of claim 25, wherein the quantity of allopurinol is sufficient to provide a patient with at least 0.25 mg allopurinol per kg body weight.

27. The formulation of claim 26, wherein the quantity of allopurinol is sufficient to provide a patient with 1 mg allopurinol per kg body weight.

28. The formulation of claim 24, wherein the inhibitor of uric acid formation comprises a compound derived from the plant Uncaria tomentosa.

29. The formulation of claim 24, wherein the inhibitor of uric acid formation comprises a compound derived from cherries.

30. The formulation of claim 24, wherein the omega-3 fatty acid is supplied via fish oil.

31. The formulation of claim 24, wherein the omega-3 fatty acid is supplied in a dose that provides a patient with 1% of daily caloric intake in omega-3 fatty acid.

32. The formulation of claim 24, wherein the omega-3 fatty acid is supplied in a dose that provides a patient with 2% of daily caloric intake in omega-3 fatty acid.

33. A formulation for minimizing inflammation related damage from a traumatic injury to a person comprising:
(a) an inhibitor of uric acid formation, (b) an omega-3 fatty acid, and (c) an antioxidant or antioxidant formulation separate from the omega-3 fatty acid.

34. The formulation of claim 33, wherein the antioxidant or antioxidant formulation separate from the omega-3 fatty acid is able to cross the blood-brain barrier.

35. The formulation of claim 34, wherein the antioxidant or antioxidant formulation separate from the omega-3 fatty acid comprises alpha lipoic acid.

36. The formulation of claim 35, wherein the alpha lipoic acid is supplied in a dose that provides a patient with 5 mg alpha lipoic acid per kg body weight.

37. The formulation of claim 35, wherein the alpha lipoic acid is supplied in a dose that provides a patient with 10 mg alpha lipoic acid per kg body weight.

38. The formulation of claim 33, wherein the antioxidant or antioxidant formulation separate from the omega-3 fatty acid comprises resveratrol.

39. The formulation of claim 33, wherein the antioxidant or antioxidant formulation separate from the omega-3 fatty acid comprises curcumin.

40. The formulation of claim 33, wherein the antioxidant or antioxidant formulation separate from the omega-3 fatty acid comprises acetyl-L-carnitine arginate.
